(19) United States
(12) Patent Application Publication
(10) Pub. No.: US 2023/0237646 A1
(43) Pub. Date: Jul. 27, 2023

(54) Radiomic Biomarker Determination Method and System for Assessment of the Risk of Metabolic Diseases

(71) Applicant: Ruijin Hospital, Shanghai Jiao Tong University School of Medicine, Shanghai (CN)

(72) Inventors: Guang Ning, Shanghai (CN); Jiqiu Wang, Shanghai (CN); Jie Hong, Shanghai (CN); Juan Shi, Shanghai (CN); Guoqing Bao, Shanghai (CN)

(21) Appl. No.: 17/582,469

(22) Filed: Jan. 24, 2022

(51) Int. Cl. G06T 7/00; A61B 6/03; A61B 6/00; G06T 7/11; G06T 11/00; G06V 10/44; G06K 9/62; G06V 10/82; G06V 10/50

(52) U.S. Cl. CPC G06T 7/0012; A61B 6/032; A61B 6/5205; A61B 6/5217; G06T 7/11; G06T 11/008; G06V 10/44; G06K 9/6256; G06V 10/82; G06V 10/50; G06V 2201/03; G06T 2207/10081; G06T 2207/30004; G06T 2207/20081; G06T 2207/20084

(57) ABSTRACT

A radiomic biomarker determination method and system for assessment of the risk of metabolic diseases. The method includes: obtaining abdominal & pelvic volumetric computed tomography (CT) scan from the given subject; determining the fat area to be analyzed from the CT scan, separating visceral fat using an image segmentation method, and normalizing the visceral fat area under physical scale; extracting N imaging features of the visceral fat; selecting n optimal imaging features from the N candidate features; dividing the normalized visceral fat area into multiple visceral fat blocks with equal thickness; extracting n corresponding optimal imaging features from each visceral fat block, named as block imaging features; and determining the representative visceral fat block from the candidate blocks and taking the representative visceral fat block and the (block) imaging features extracted from the representative visceral fat block as radiomic biomarkers.

### TECHNICAL FIELD

The present disclosure is related to the technical fields of radiomics, machine learning, and medical image processing, and in particular, a radiomic biomarker determination method and system for assessment of the risk of metabolic diseases.

### BACKGROUND ART

As a new image analysis technology, radiomics has been successfully applied to improve the diagnosis and prognosis of several cancers in recent years. Using this technology, high-throughput texture features can be extracted from medical images. These imaging features are hidden in medical big data and are usually not perceivable by the naked eyes. However, these deep features may provide more or higher discriminative information for the prevention, diagnosis, and even treatment of diseases. Recent studies have shown that quantitative and radiomic features constructed from the abdominal region demonstrated certain capabilities for the diagnosis of metabolic-related diseases. For example, Kim et al. measured the areas of adipose tissue from abdominal CT scans and suggested that the deep subcutaneous fat is related to the increased inflammation and oxidative stress, which may serve as one of the pathogenic factors of metabolic syndrome. Lu et al. constructed radiomic features from three abdominal organs using CT scans and they found that imaging features obtained from the pancreas demonstrated higher performance for discrimination of early type 2 diabetes.

However, qualitative factors, especially the texture of visceral fat tissues, in the evaluation of metabolic diseases are largely unexplored. It is thus necessary to develop a non-invasive and cost-effective method to obtain radiomic biomarkers from the texture of fat tissues which can be used to assess the risk of metabolic diseases.

### SUMMARY

The objective of the present disclosure is to provide a radiomic biomarker determination method and system for non-invasive, low-radiation, and cost-effective assessment of the risk of metabolic diseases.

To implement the given objectives, the present disclosure provides the following solutions.

A radiomic biomarker determination method for assessment of the risk of metabolic diseases include:

obtaining abdominal & pelvic volumetric computed tomography (CT) scan from the given subject;

determining the fat area to be analyzed in the CT scan according to anatomical positions of the diaphragm and the pubic symphysis;

separating visceral fat from the determined fat area with an image segmentation method;

extracting N imaging features from the visceral fat;

selecting n optimal imaging features from the N candidate features based on their Gini coefficients in the determination of presence or absence of metabolic syndrome, where N>n;

normalizing the visceral fat area under the physical scale, and dividing the normalized visceral fat area into multiple visceral fat blocks with equal thickness;

extracting n corresponding optimal imaging features from each visceral fat block, named as block imaging features; and

determining the representative visceral fat block from the candidate blocks according to their cross-validation performance for assessment of metabolic syndrome, and taking the (block) imaging features extracted from the representative visceral fat block as radiomic biomarkers.

Optionally, the imaging features may include first-order intensity, gray-level co-occurrence matrix (GLCM), gray-level run-length matrix (GLRLM), gray-level size zone matrix (GLSZM), and wavelet features.

Optionally, the normalization and fat area division process may specifically include:

normalizing the visceral fat area under the physical scale based on the voxel size, and dividing the normalized visceral fat area into the multiple visceral fat blocks with equal thickness.

Optionally, the radiomic biomarker determination method for assessment of the risk of metabolic diseases may further include:

assessing the block imaging features extracted from the representative visceral fat block on the test data set with a pre-trained model.

Optionally, the procedure of the performance assessment may include:

obtaining the training data set, where training samples are CT scans obtained from human candidates;

for each sample CT scan, determining the fat area to be analyzed according to anatomical positions of the diaphragm and the pubic symphysis;

separating visceral fat from the determined fat area with an image segmentation method;

extracting N imaging features from the visceral fat;

selecting n optimal imaging features from the N candidate features based on their Gini coefficients in the determination of presence or absence of the metabolic syndrome;

normalizing the visceral fat area under the physical scale, and dividing the normalized visceral fat area into multiple visceral fat blocks with equal thickness;

extracting n corresponding optimal imaging features from each sample visceral fat block, named as block imaging features;

determining the representative visceral fat block from the candidate sample blocks according to their cross-validation performance; and

iteratively training a feed-forward neural network with 10 block imaging features extracted from the representative visceral fat block to obtain the assessment model; and the trained model are assessed on the test set, where performance indicators include precision, recall, f1 score, accuracy, and area under the curve (AUC), for assessment of the risk of metabolic-related diseases.

Optionally, the neural network model may include five fully connected layers, two Gaussian noise layers, a random dropout layer, and a SoftMax layer.

To implement the objectives, the present disclosure further provides the following solutions.

A radiomic biomarker determination system for assessment of the risk of metabolic diseases include:

an image acquisition unit configured to obtain abdominal & pelvic CT scans from the given subjects;

a fat division unit, attached to the image acquisition unit, and configured to determine the fat area to be analyzed based on anatomical positions of the diaphragm and the pubic symphysis;

a segmentation unit, attached to the fat division unit, and configured to separate visceral fat from the determined fat area using an image segmentation method;

a feature extraction unit, attached to the segmentation unit, and configured to extract N imaging features from the visceral fat;

an optimal feature determination unit, attached to the feature extraction unit, and configured to select n optimal imaging features from the N candidate features based on their Gini coefficients in the determination of presence and absence of metabolic syndrome, where N>n;

a fat block division unit, attached to the segmentation unit, and configured to normalize the visceral fat area under the physical scale, and divide the normalized visceral fat area into multiple visceral fat blocks with equal thickness;

a block feature extraction unit, attached to the optimal feature determination unit and the fat block division unit respectively, and configured to extract n corresponding optimal imaging features from each visceral fat block, named as block imaging features; and

a radiomic biomarker determination unit, attached to the block feature extraction unit, and configured to determine the representative visceral fat block from the candidate visceral fat blocks according to their cross-validation performance, and the (block) imaging features extracted from the representative visceral fat block are taken as radiomic biomarkers.

According to the specific embodiments provided by the present disclosure, the present disclosure provides the following technical effects: 1) the fat area to be analyzed is determined from the CT scan; 2) the visceral fat is separated using an image segmentation method, and N imaging features of the visceral fat are extracted with radiomics; 3) n optimal imaging features are selected from the N candidate features based on their Gini coefficients or their capabilities in determination of the presence and absence of the metabolic syndrome; 4) the visceral fat area normalized under the physical scale is divided into multiple visceral fat blocks with equal thickness; 5) n corresponding optimal block imaging features are extracted from each visceral fat block, and the representative visceral fat block is determined from the candidate blocks according to their cross-validation performance; 6) the block imaging features extracted from the representative visceral fat block are used as the radiomic biomarkers. Only the representative visceral fat block area is required to obtain (scanned), and the radiomic biomarkers extracted from the representative block can be used for assessment of the risk of metabolic-related diseases, such as metabolic syndrome, insulin resistance, and visceral obesity, without blood tests, which is non-invasive, low-radiation, and cost-effective.

### BRIEF DESCRIPTION OF THE DRAWINGS

To describe the technical solutions in the embodiments of the present disclosure or the prior art more clearly, the figure illustrations required for the embodiments are briefly described below. Apparently, the accompanying drawings in the following descriptions show merely some embodiments of the present disclosure, and a person of ordinary skill in the art may still derive other accompanying drawings from these accompanying drawings without creative efforts.

FIG. 1 is a flowchart of a radiomic biomarker determination method for assessment of the risk of metabolic diseases in the present disclosure; and

FIG. 2 is a schematic diagram of the module structure for the radiomic biomarker determination system in the assessment of the risk of metabolic diseases in the present disclosure.

### REFERENCE NUMERALS

Image acquisition unit-1, fat division unit-2, segmentation unit-3, feature extraction unit-4, optimal feature determination unit-5, fat block division unit-6, block feature extraction unit-7, radiomic biomarker determination unit-8, and assessment unit-9.

### DETAILED DESCRIPTION OF THE EMBODIMENTS

The technical solutions in the embodiments of the present disclosure are clearly and completely described in the below figure illustrations. Apparently, the described embodiments are merely a part rather than all of the embodiments of the present disclosure. All other embodiments obtained by a person of ordinary skill in the art on the basis of the embodiments of the present disclosure without creative efforts shall fall within the protection scope of the present disclosure.

The objective of the present disclosure is to provide a radiomic biomarker determination method and system for assessment of the risk of metabolic diseases, which determines the radiomic biomarkers through visceral fat separation, normalization, and feature extraction, as well as representative fat block determination. Only the representative visceral fat block area is required to obtain (scanned), and the radiomic biomarkers extracted from the representative block can be used for assessment of the risk of metabolic-related diseases without blood tests, which is non-invasive, low-radiation, and cost-effective.

To achieve the abovementioned objectives, and to make features and advantages of the present disclosure clearer and more comprehensible, the present disclosure will be further described in detail below with illustrations of the implementation details.

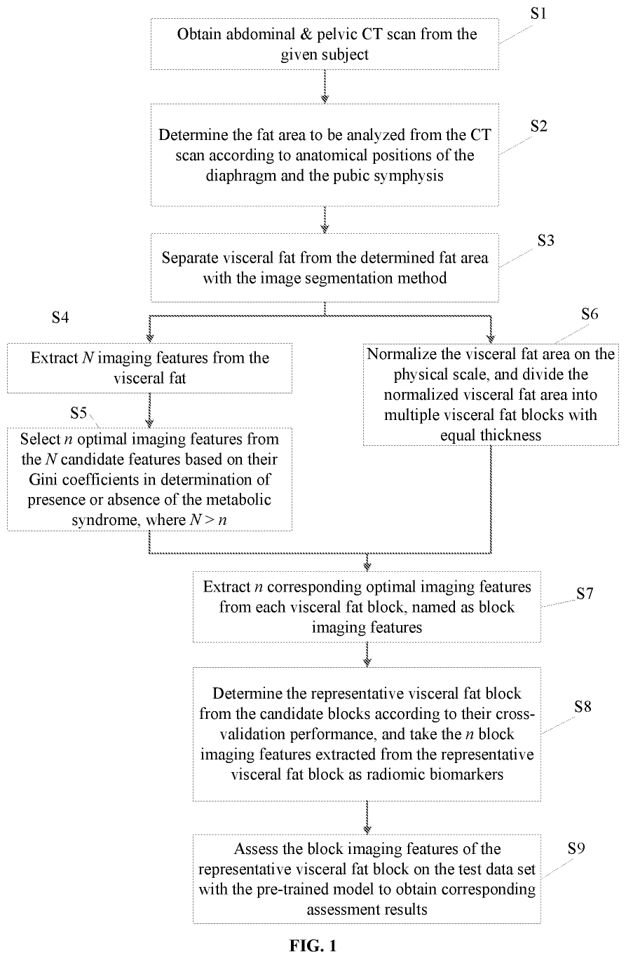

As shown in FIG. 1, a radiomic biomarker determination method for assessment of the risk of metabolic diseases in the present disclosure includes the following steps.

S1: abdominal & pelvic CT scan is obtained from the given subject.

S2: the fat area to be analyzed in the CT scan is determined according to anatomical positions of the diaphragm and the pubic symphysis.

S3: visceral fat is separated from the determined fat area by using an image segmentation method. In the present embodiment, the image segmentation method determines visceral fat and subcutaneous fat tissues based on signal intensity, morphology, and connectivity.

S4: N imaging features are extracted from the visceral fat. Specifically, radiomics technology is used to extract 377 imaging features from the visceral fat. The imaging features include first-order intensity, GLCM, GLRLM, GLSZM, and wavelet features.

S5: n optimal imaging features are selected from the N candidate features based on their Gini coefficients in the determination of the presence and absence of metabolic syndrome. N>n. Specifically, n (n=10) imaging features that are most closely related to the metabolic syndrome or most effective in predicting the metabolic syndrome are selected from N (N=377) imaging features.

S6: the visceral fat area is normalized under the physical scale, and the normalized visceral fat area is divided into multiple visceral fat blocks with equal thickness. In the present embodiment, the division is based on voxel size. Specifically, there are 10 fat blocks, and each fat block has about 51 slices or has a thickness of about 51 mm on average.

S7: n corresponding optimal imaging features are extracted from each visceral fat block, named as block imaging features.

S8: the representative visceral fat block is determined from the candidate blocks according to their cross-validation performance, and the n block imaging features extracted from the representative visceral fat block are taken as radiomic biomarkers. In order to be more suitable for clinical use, i.e., reducing radiation and saving costs, the present disclosure further explores the representative fat blocks instead of CT scans of the entire chest, abdomen, and pelvic. The representative visceral fat block is determined as regions around the umbilical level in the present embodiment.

To guarantee the robustness of the radiomic biomarker determination method in the assessment of the risk of metabolic diseases, the present disclosure further includes the following step.

S9: the block imaging features extracted from the representative visceral fat block are assessed on the test data set using the pre-trained model. The assessment criteria include precision, recall, f1 score, accuracy, and area under the curve (AUC).

The procedure for establishing the assessment model includes the following steps.

The training data set containing abdominal & pelvic CT scans are obtained from human candidates.

For each CT scan, the fat area to be analyzed is determined according to anatomical positions of the diaphragm and the pubic symphysis.

Visceral fat is separated from the determined fat area using an image segmentation method.

N sample imaging features are extracted from the visceral fat.

n optimal sample imaging features are selected from the N candidate sample features based on their Gini coefficients in the determination of the presence and absence of the metabolic syndrome.

The visceral fat area is normalized under physical scale, and the normalized visceral fat area is divided into multiple visceral fat blocks with equal thickness.

n corresponding optimal block imaging features are extracted from each visceral fat block, named as block imaging features.

The representative visceral fat block is determined from the candidate sample blocks according to their cross-validation performance.

A feed-forward neural network is iteratively trained with the n block imaging features extracted from the representative visceral fat block. The trained model is assessed on the test set. Performance indicators for assessment of the risk of metabolic-related diseases include precision, recall, f1 score, accuracy, and AUC.

Further, the neural network model includes five fully connected layers (with 1024, 512, 256, 128, and 64 nodes respectively), two Gaussian noise layers, a random dropout layer, and a SoftMax layer.

The Gaussian noise layer and the random dropout layer improve the generalization of the neural network by introducing input variances and reducing the dependence among neurons.

L2 regularization is used to constrain the neural network parameters to reduce overfitting.

He-normalization, as a type of network initialization methods, is used to initialize the network weight.

Hyperparameter search is conducted under 10-fold cross-validation.

In order to explore the radiomic biomarker that can be used to assess the risk of metabolic-related diseases, in the present disclosure, 675 volunteers who conducted abdominal and pelvic CT scanning were enrolled. 80% of the sample CT scans are used as the training set and 20% of them are for testing. The texture features of visceral fat are extracted with image segmentation and radiomics, and the radiomic biomarker is identified with machine learning.

The radiomic biomarkers extracted from the representative visceral fat block (around the umbilical level) achieved precision, recall, f1 score, and accuracy of 0.847, 0.812, 0.817, and 0.812 respectively, for the assessment of the metabolic syndrome in the present disclosure, which is superior compared to features extracted from other parts of the CT scan.

The neural network achieved higher AUC and accuracy performance in predicting the prevalence of metabolic syndrome, i.e., 0.888 and 83.0% respectively on the test set, which is superior compared to traditional machine learning methods such as random forests (RF; 0.864 and 75.6%) and support vector machine (SVM; 0.861 and 80.0%).

In combination gender with the top 10 radiomic biomarkers extracted from visceral fat of the representative fat block, the neural network demonstrated AUC performances of 0.872, 0.888, 0.961, and 0.947 respectively for predicting the prevalence of insulin resistance, metabolic syndrome, central obesity, and visceral obesity.

Wavelet HHH GLRLM RunEntropy is one of the identified radiomic biomarkers in the present disclosure, which achieved superior AUCs of 0.819 and 0.816 respectively for diagnosis of metabolic syndrome and insulin resistance compared to body block index (BMI) and waist circumference. In addition, the present disclosure extracted radiomic biomarkers at 10 random locations around the umbilical level and performed comparing analyses, which verified the robustness of the selected imaging features and the location of the representative fat block. CT scanning around the umbilical level is sufficient to non-invasively assess the metabolic syndrome, insulin resistance, and visceral obesity, which can minimize the radiation and cost, and obviates the need for blood tests.

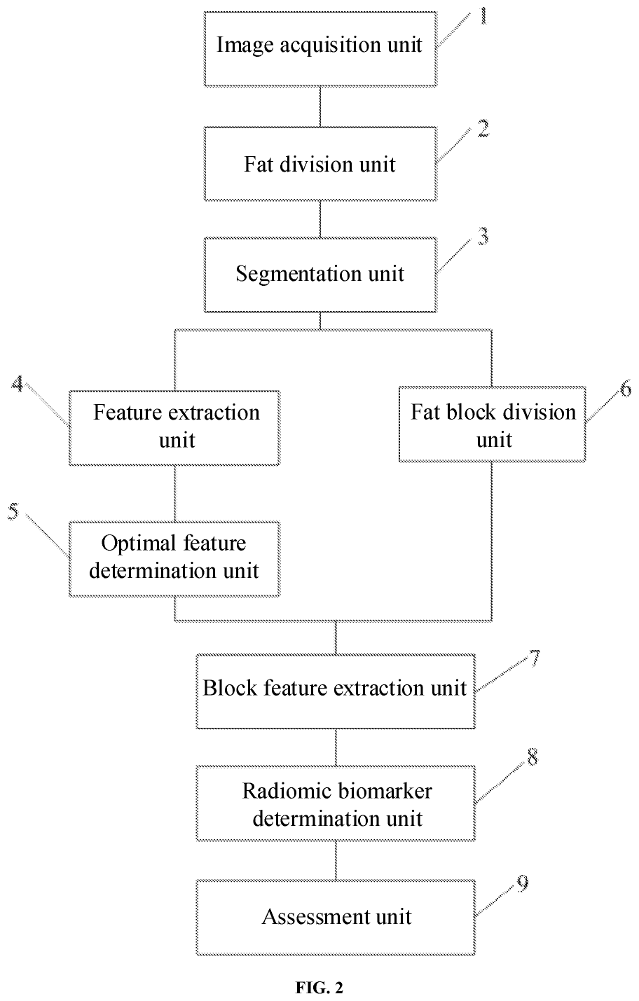

As shown in FIG. 2, a radiomic biomarker determination system for assessment of the risk of metabolic diseases in the present disclosure includes an image acquisition unit 1, a fat division unit 2, a segmentation unit 3, a feature extraction unit 4, an optimal feature determination unit 5, a fat block division unit 6, a block feature extraction unit 7, and a radiomic biomarker determination unit 8.

The image acquisition unit 1 is configured to obtain volumetric CT scans from the given subjects.

The fat division unit 2 is attached to the image acquisition unit 1 and is configured to determine the fat area to be analyzed in the CT scan according to anatomical positions of the diaphragm and the pubic symphysis.

The segmentation unit 3 is attached to the fat division unit 2 and is configured to separate visceral fat from the determined fat area using an image segmentation method.

The feature extraction unit 4 is attached to segmentation unit 3 and is configured to extract N imaging features from the visceral fat.

The optimal feature determination unit 5 is attached to the feature extraction unit 4 and is configured to select n optimal imaging features from the N candidate features based on their Gini coefficients in the determination of presence and absence of metabolic syndrome.

The fat block division unit 6 is attached to the segmentation unit 3 and is configured to normalize the visceral fat area under physical scale and divide the normalized visceral fat area into multiple visceral fat blocks with equal thickness.

The block feature extraction unit 7 is attached to the optimal feature determination unit 5 and the fat block division unit 6 respectively and is configured to extract n corresponding optimal imaging features from each visceral fat block, named as block imaging features.

The radiomic biomarker determination unit 8 is attached to the block feature extraction unit 7 and is configured to determine the representative visceral fat block from the candidate blocks according to their cross-validation performance, and imaging features extracted from the representative visceral fat block are taken as radiomic biomarkers.

Further, the radiomic biomarker determination system for assessment of the risk of metabolic diseases further includes an assessment unit 9. The assessment unit 9 is attached to the radiomic biomarker determination unit 8 and is configured to assess the imaging features extracted from the representative visceral fat block on the test data set using the pre-trained model. The assessment criteria include precision, recall, f1 score, accuracy, and AUC.

The radiomic biomarker determination system of the present disclosure has the same beneficial effects as the abovementioned radiomic biomarker determination method, and the beneficial effects are not repeated herein.

Each embodiment of this specification is described in a progressive manner, each embodiment focuses on its unique points, and the same and similar parts between the embodiments may refer to each other. Since the system corresponds to the method disclosed in the embodiments, the descriptions of the system are not detailed, and reference can be made to the method description.

In this specification, several specific embodiments are used for the illustration of the principles and implementations of the present disclosure. The description of the foregoing embodiments is used to help illustrate the method of the present disclosure and the core ideas thereof. In addition, persons of ordinary skill in the art can make various modifications in terms of specific implementations and the scope of application in accordance with the ideas of the present disclosure. In conclusion, the content of this specification shall not be construed as a limitation to the present disclosure.

What is claimed is:

1. A radiomic biomarker determination method for assessment of the risk of metabolic diseases, comprising:
obtaining abdominal & pelvic volumetric computed tomography (CT) scan from the given subject;
determining the fat area to be analyzed from the CT scan according to anatomical positions of the diaphragm and the pubic symphysis;
separating visceral fat from the determined fat area with an image segmentation method;
extracting N imaging features from the visceral fat;
selecting n optimal imaging features from the N candidate features based on their Gini coefficients in the determination of presence and absence of metabolic syndrome, wherein N>n;
normalizing the visceral fat area under physical scale, and dividing the normalized visceral fat area into multiple visceral fat blocks with equal thickness;
extracting n corresponding optimal imaging features from each visceral fat block, named as block imaging features; and
determining the representative visceral fat block from the candidate blocks according to their cross-validation performance, and taking the imaging features extracted from the representative visceral fat block as radiomic biomarkers.

2. The radiomic biomarker determination method for assessment of the risk of metabolic diseases according to claim 1, wherein the imaging features comprise first-order intensity, gray-level co-occurrence matrix (GLCM), gray-level run-length matrix (GLRLM), gray-level size zone matrix (GLSZM), and wavelet features.

3. The radiomic biomarker determination method for assessment of the risk of metabolic diseases, according to claim 1, is composed of a procedure for visceral fat area normalization under the physical scale, and dividing the normalized visceral fat area into the multiple visceral fat blocks with equal thickness.

4. The radiomic biomarker determination method for assessment of the risk of metabolic diseases according to claim 1, further comprising:
assessing the block imaging features extracted from the representative visceral fat block on the test data set with the pre-trained model.

5. The radiomic biomarker determination method for assessment of the risk of metabolic diseases according to claim 4, the procedure for establishing the assessment model comprises:
obtaining the training data set, wherein training samples are abdominal & pelvic CT scans obtained from human candidates;
for each sample CT scan, determining the fat area to be analyzed according to anatomical positions of the diaphragm and the pubic symphysis;
separating visceral fat from the determined fat area with the image segmentation method;
extracting N sample imaging features from the visceral fat;
selecting n optimal sample imaging features from the N candidate sample features based on their Gini coefficients in the determination of presence and absence of the metabolic syndrome;
normalizing the visceral fat area under physical scale, and dividing the normalized visceral fat area into multiple visceral fat blocks with equal thickness;
extracting n corresponding optimal imaging features from each visceral fat block, named as block imaging features;
determining the representative visceral fat block from the candidate blocks according to their cross-validation performance; and
iteratively training a feed-forward neural network with the n block imaging features extracted from the representative visceral fat block; and the trained model is assessed on the test set, wherein performance indicators include precision, recall, f1 score, accuracy, and area under the curve (AUC), for the assessment of the risk of metabolic-related diseases.

6. The radiomic biomarker determination method for assessment of the risk of metabolic diseases according to claim 4, wherein the neural network model comprises five fully connected layers, two Gaussian noise layers, a random dropout layer, and a SoftMax layer.

7. The radiomic biomarker determination method for assessment of the risk of metabolic diseases according to claim 5, wherein the neural network model comprises five fully connected layers, two Gaussian noise layers, a random dropout layer, and a SoftMax layer.

8. A radiomic biomarker determination system for assessment of the risk of metabolic diseases, comprising:
an image acquisition unit configured to obtain abdominal & pelvic volumetric CT scan from the given subject;
a fat division unit, attached to the image acquisition unit, and configured to determine the fat area to be analyzed according to anatomical positions of the diaphragm and the pubic symphysis;
a segmentation unit, attached to the fat division unit, and configured to separate visceral fat from the determined fat area using an image segmentation method;
a feature extraction unit, attached to the segmentation unit, and configured to extract N imaging features from the visceral fat;
an optimal feature determination unit, attached to the feature extraction unit, and configured to select n optimal imaging features from the N candidate features based on their Gini coefficients in the determination of presence and absence of metabolic syndrome, wherein N>n;
a fat block division unit, attached to the segmentation unit, and configured to normalize the visceral fat area under physical scale, and divide the normalized visceral fat area into multiple visceral fat blocks with equal thickness;
a block feature extraction unit, attached to the optimal feature determination unit and the fat block division unit respectively, and configured to extract n corresponding optimal imaging features from each visceral fat block, named as block imaging features; and
a radiomic biomarker determination unit, attached to the block feature extraction unit, and configured to determine the representative visceral fat block from the candidate blocks according to their cross-validation performance, and the imaging features extracted from the representative visceral fat block are taken as radiomic biomarkers.

9. The radiomic biomarker determination system for assessment of the risk of metabolic diseases according to claim 8, wherein the imaging features comprise first-order intensity, GLCM, GLRLM, GLSZM, and wavelet features.

10. The radiomic biomarker determination system for assessment of the risk of metabolic diseases according to claim 8, further comprising:
an assessment unit, attached to the radiomic biomarker determination unit, and configured to assess the block imaging features extracted from the representative visceral fat block on the test data set using the pre-trained model.

11. The radiomic biomarker determination system for assessment of the risk of metabolic diseases according to claim 10, wherein the assessment model comprises five fully connected layers, two Gaussian noise layers, a random dropout layer, and a SoftMax layer.
